(19) United States
(12) Patent Application Publication
(10) Pub. No.: US 2024/0368074 A1
(43) Pub. Date: Nov. 7, 2024

(54) UREA SYNTHESIS FROM GREENHOUSE GAS AND NITROGEN VIA TRANSITION METAL-FREE CATALYSIS OVER GRAPHITIC CARBON NITRIDE

(71) Applicant: KING FAISAL UNIVERSITY, AL-AHSA (SA)

(72) Inventors: HASSAN H. HAMMUD, AL-AHSA (SA); KHURSHID AYUB, AL-AHSA (SA); MUHAMMAD YAR, AL-AHSA (SA); NADEEM SADIQ SHEIKH, AL-AHSA (SA); RANJITH KUMAR KARNATI, AL-AHSA (SA); DOLAYL ESSA AL-HUDAIRI, AL-AHSA (SA)

(21) Appl. No.: 18/141,994

(22) Filed: May 1, 2023

(51) Int. Cl. C07C 273/02; C07C 273/16; C25B 3/09; C25B 3/26; C25B 3/29; C25B 11/091

(52) U.S. Cl. CPC C07C 273/02; C07C 273/16; C25B 3/09; C25B 3/26; C25B 3/29; C25B 11/091

(57) ABSTRACT

A urea synthesis from the reaction of readily available greenhouse gas carbon dioxide (CO₂) and nitrogen gas (N₂) via transition metal-free catalysis on dual boron-doped (2B) two-dimensional (2D) graphitic carbon nitride is provided.

### BACKGROUND

### 1. Field

The disclosure of the present patent application relates to urea synthesis from a greenhouse gas (CO₂) and nitrogen (N₂) gas via transition metal-free catalysis on graphitic carbon nitride, such as dual boron doped graphitic carbon nitride.

### 2. Description of the Related Art

Due to commercial utilization of urea, the prospect of any innovative and environmentally benign process towards the synthesis of urea will have an enormous advantage. Owing to 46% nitrogen content in urea, 90+% of industrial production of urea is dedicated for the fertilizer industry to meet the daunting challenges of global food security as well as other areas and industries. Commercially, urea production is primarily carried out by the reaction of liquid ammonia (NH₃) and liquid carbon dioxide (CO₂) at elevated temperatures and high pressures. In addition, the ammonia required for urea synthesis is chiefly obtained through nitrogen fixation by a reduction reaction. These are complex, multiple step synthetic processes that are less efficient due to energy and logistic requirements. Application of several transition metal-based materials for urea synthesis is already reported in the literature. Nevertheless, these processes are known to suffer with inefficient nitrogen fixation and a low production rate (mmol g−1 h−1). Moreover, involvement of highly toxic transition metals makes these process unattractive and warrant development of innovative approaches.

For examples of past technology processes, U.S. Pat. No. 4,542,006 discloses that a catalytic process has been found to produce urea or its equivalent, NH₄NCO, under relatively mild pressure and temperature conditions. This process entails flowing a gas mixture containing NOₓ, CO, and a source of hydrogen such as H₂ or H₂C over a hydrogenation catalyst such as platinum.

Furthermore, US Published Patent Application No. 2017/0166518 discloses a method for increasing the capacity of a urea production complex, the method comprising a step of adding to an existing urea production complex a CO₂ production unit, which unit employs a CO₂ production method comprising: i) subjecting a hydrocarbon feed to short contact time catalytic partial oxidation (SCT-CPO) to produce a first gas mixture comprising H₂, CO and CO₂, ii) subjecting said first gas mixture to a water gas shift reaction yielding a second gas mixture, iii) separating CO₂ from said second gas mixture yielding a purified CO₂ stream and a hydrogen containing stream, and subsequently iv) reacting said purified CO₂ stream with ammonia from the ammonia production unit to produce urea.

Additionally, US Published Patent Application No. 2011/0114503 discloses methods and systems for electrochemical production of urea. A method may include, but is not limited to, steps (A) to (B). Step (A) may introduce carbon dioxide and NOₓ to a solution of an electrolyte and a heterocyclic catalyst in an electrochemical cell. The divided electrochemical cell may include an anode in a first cell compartment and a cathode in a second cell compartment. The cathode may reduce the carbon dioxide and the NOₓ into a first sub-product and a second sub-product, respectively. Step (B) may combine the first sub-product and the second sub-product to produce urea.

Further, US Published Patent Application No. 2016/0233487 discloses graphitic carbon nitride materials that are useful in electrochemical cells such as Lithium-Sulfur batteries. Also disclosed are lithium-sulfur batteries designed to incorporate these materials and methods of manufacturing the same. Batteries that include this material exhibit increased electrode kinetics of the lithium-sulfur electrochemical couple, phenomena that improve the specific capacity, usable lifetime, and other desirable characteristics of these batteries. The disclosed graphitic carbon nitride (g-CN) materials are non-toxic and can be readily made from sustainable feed stocks such as dicyandiamide, urea, melamine, and many other nitrogen rich molecules. In certain embodiments, the graphitic carbon nitride is doped with at least one of: Sulfur, Carbon, Phosphorus, or Boron.

Additional prior art generally discloses urea production, and production of boron doped graphitic carbon nitride that may be made with urea and used in electrochemical cells. However, urea synthesis from a greenhouse gas and nitrogen gas via non-toxic transition metal-free catalysis is desired for less toxicity, more efficient nitrogen fixation and a higher production rate.

### SUMMARY

The presently disclosed subject matter relates to a process for urea synthesis through utilization of atmospheric gases. It is envisaged to produce urea by the reaction of a readily available greenhouse gas (CO₂) and nitrogen gas (N₂) on a two dimensional graphitic carbon nitride, such as a dual boron-doped (2B) two-dimensional (2D) graphitic carbon nitride (2B@CN). Accordingly, the present synthesis methods can involve binding of two nitrogen atoms to the two boron atoms of a 2B@CN complex followed by coupling of carbon dioxide (CO₂) to one of the two boron atoms. The resultant product can be hydrogenated and then subjected to a reduction reaction to produce urea on the dual boron-doped 2D graphitic carbon nitride. This protocol tends to suppress hydrogen evolution with high Faradic efficiency leading to a high urea formation rate (mmol g⁻¹ h⁻¹). This process provides a novel commercially viable method for the production of urea which is based on transition metal free catalysis.

The current subject matter approach to prepare urea is based on transition metal free catalysis which involves fixation of dinitrogen using two atoms of a cheap, readily available metalloid such as boron. This is in sharp contrast to existing protocols for the synthesis of urea where precious metals such as gold and platinum are employed in catalytic processes. A metalloid atom has filled and vacant p-orbitals which behave like the d-orbitals of the transition metal atoms. This means the non-toxic metalloid mimics the reactivity pattern of the transition metal and serves as a safe replacement for toxic transition metal.

Accordingly, in one embodiment, the present subject matter relates to a method of synthesizing urea, the method comprising providing a two dimensional (2D) graphitic carbon nitride scaffold; binding two boron atoms to the 2D graphitic carbon nitride scaffold to produce a dual boron-doped 2D graphitic carbon nitride (2B@CN) catalyst, electrocatalyst, or complex; reacting carbon dioxide (CO₂) and nitrogen gas (N₂) with the dual boron-doped (2B) two dimensional (2D) graphitic carbon nitride (2B@CN) to produce urea on the dual boron-doped 2D graphitic carbon nitride; and separating the urea from the dual boron-doped 2D graphitic carbon nitride to obtain urea as a final product.

In one embodiment, the present subject matter relates to a method of urea synthesis by the reaction of readily available greenhouse gas (CO₂) and dinitrogen/nitrogen gas (N₂) on dual boron-doped (2B) 2D graphitic carbon nitride (C₃N₄).

In an embodiment, the present subject matter relates to a method of urea synthesis also involving adsorption of inert nitrogen gas to the two boron atoms of a 2B@CN complex followed by coupling of carbon dinoxide (CO₂) thereto.

In an embodiment, the present subject matter relates to a method of urea synthesis that is also based on transition metal free catalysis.

These and other features of the present subject matter will become readily apparent upon further review of the following specification.

### BRIEF DESCRIPTION OF THE DRAWINGS

FIG. 1 shows a synthetic process for preparing urea from dual boron doped graphitic carbon nitride (C₃N₄) using CO₂ and N₂.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ≥10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The presently disclosed subject matter relates to a process for urea synthesis through utilization of atmospheric gases. It is envisaged to produce urea by the reaction of a readily available greenhouse gas (CO₂) and nitrogen gas (N₂) on dual boron-doped (2B) two dimensional (2D) graphitic carbon nitride (C₃N₄). This innovative approach involves adsorption of inert nitrogen on the surface of a 2B@CN complex followed by coupling of carbon dioxide (CO₂) on one of the two nitrogen atoms. Consequently, urea is produced on the dual boron-doped 2D graphitic carbon nitride by a reduction reaction of the carbon dioxide and the two nitrogen atoms. The urea can then be separated from the 2B@CN catalyst, thereby obtaining a urea product. This protocol tends to suppress hydrogen evolution with high Faradic efficiency leading to a high urea formation rate (mmol g⁻¹ h⁻¹). This process provides a novel commercially viable method for producing urea which is based on transition metal free catalysis. That is to say, an aspect of the present methods of urea synthesis is that no transition metals are required to catalyze the urea synthesis.

In one embodiment, the present subject matter relates to a method of synthesizing urea, the method comprising providing a two dimensional (2D) graphitic carbon nitride scaffold; binding two boron atoms to the 2D graphitic carbon nitride to produce a dual boron-doped 2D graphitic carbon nitride (2B@CN); reacting carbon dioxide (CO₂) and nitrogen gas (N₂) with the dual boron-doped (2B) two dimensional (2D) graphitic carbon nitride (2B@CN) to produce urea on the dual boron-doped 2D graphitic carbon nitride; and separating the urea from the dual boron-doped 2D graphitic carbon nitride to obtain urea as a final product.

In an embodiment of the present methods, once the boron atoms are bound to the 2D graphitic carbon nitride, two nitrogen atoms from the nitrogen gas can in turn be bound to the two boron atoms. In a further embodiment, the carbon dioxide (CO₂) can then be coupled to one of the two nitrogen atoms which are bound to the two boron atoms.

In an embodiment, the present subject matter also relates to a method wherein the urea produced on the dual boron-doped 2D graphitic carbon nitride is produced by a reduction reaction of the carbon dioxide and the two nitrogen atoms.

In another embodiment, the two-dimensional graphitic carbon nitride scaffold used in the present methods can be prepared by pyrolysis of an aromatic carbon nitrogen rich source under a nitrogen atmosphere. In this regard, a metal complex catalyst can optionally be present in this pyrolysis step. When present, the metal complex catalyst can include, by way of non-limiting example, a metal selected from the group consisting of cobalt, nickel, iron, and combinations thereof. In an embodiment, the metal complex catalyst provides an additional source of carbon and nitrogen for providing the two-dimensional graphitic carbon nitride scaffold. In a further embodiment, the binding of the two boron atoms to the 2D graphitic carbon nitride can occur during the pyrolysis.

In another embodiment, following separation of the urea from the dual boron-doped 2D graphitic carbon nitride catalyst, the two boron atoms can be further removed from the dual boron-doped 2D graphitic carbon nitride catalyst, resulting in a two-dimensional graphitic carbon nitride scaffold that can be reused in the method of synthesizing urea.

Part of the process may include one or more hydrogenation reactions, where hydrogen is added to one of the multiple bonds present. Such a hydrogenation process is applicable to almost all types of multiple bonds and is of great importance in synthetic chemistry, particularly in the chemical industry.

The exact mechanisms of heterogeneous reactions are difficult to determine, but much interesting and helpful information has been obtained for catalytic hydrogenation. The metal catalyst is believed to act by binding the reactants at the surface of a crystal lattice. As used herein, the carbon nitride scaffold can be bound to two boron atoms (2B), with the boron atoms serving in place of a metal(s) or transition metal(s) catalysts, like nickel, for reasons stated previously-less toxicity, more efficient nitrogen fixation and a higher production rate.

The subject matter of the present application can be further understood by referring to the following examples.

### EXAMPLES

### Example 1

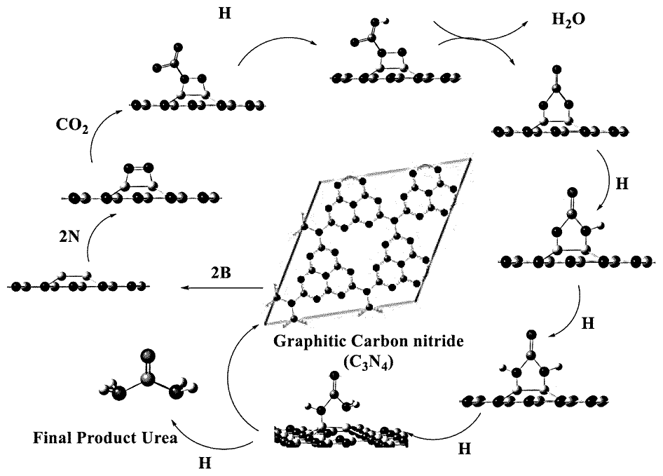

Urea synthesis from a greenhouse gas such as carbon dioxide and a nitrogen gas is based on computational studies. Based on theoretical calculations run using validated software, the results indicate that the process is practically viable, and the proposed pathway can be applied on an industrial scale. The details of the proposed study, and the synthetic scheme thereof, are described in FIG. 1. Urea synthesis from CO₂ and N₂ is primarily achievable due to 3 major observations: 1) weak adsorption of CO₂ and N₂ on the designed catalytic surface, 2) bond dissociation of N═N and C═O, and 3) reduction reaction of CO₂/N₂. All these aspects are due to the weak C—N and C-C coupling with the catalyst surface. The experimental and theoretical studies reveal that a boron based electrocatalyst shows excellent performance for C-C and C—N and coupling.

It is shown computationally that dual boron doped graphitic carbon nitride (2B@CN) is a potential electrocatalytic material for synthesis of urea from greenhouse gas (CO₂) and N₂ gas. The selected graphitic carbon nitride surface can have a high nitrogen content in its cavity, which can strongly bind boron atoms. In addition, boron contains three electrons in its valence shell and an empty p-orbital that resembles a d-orbital of the transition metals. So, the presence of the vacant p-orbital facilitates in the binding and activation of inert N₂ and CO₂, as seen in FIG. 1.

All possible steps involved in the present urea synthesis are provided in FIG. 1, starting with graphitic carbon nitride (C₃N₄). Based on the presence of weak C—N and C-C coupling on the catalyst surface, the computational calculation-based urea synthesis can be efficiently carried out on 2B@CN.

It is to be understood that the compositions, systems, and methods as described herein are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

1. A method of synthesizing urea, the method comprising providing a two dimensional (2D) graphitic carbon nitride scaffold;
binding two boron atoms to the 2D graphitic carbon nitride to produce a dual boron-doped 2D graphitic carbon nitride;
binding two nitrogen atoms to the two boron atoms;
coupling carbon dioxide to one of the two nitrogen atoms after binding of the two nitrogen atoms to the two boron atoms to provide a dual boron-doped 2D graphitic carbon nitride coupled with carbon dioxide;
hydrogenating the dual boron-doped 2D graphitic carbon nitride coupled with carbon dioxide;
reacting carbon dioxide (CO₂) and nitrogen gas (N₂) with the dual boron-doped two dimensional (2D) graphitic carbon nitride after the hydrogenating to produce urea on the dual boron-doped 2D graphitic carbon nitride; and
separating the urea from the dual boron-doped 2D graphitic carbon nitride to obtain urea as a final product.

2. (canceled)

3. (canceled)

4. (canceled)

5. The method as recited in claim 1, wherein transition metals are excluded in the urea synthesis.

6. The method as recited in claim 1, further comprising pyrolyzing an aromatic carbon nitrogen rich source under a nitrogen atmosphere to provide the two dimensional (2D) graphitic carbon nitride scaffold.

7. The method as recited in claim 6, wherein a metal complex catalyst is present in the pyrolysis step, the metal complex catalyst including a metal selected from the group consisting of cobalt, nickel, iron, and combinations thereof.

8. (canceled)

9. (canceled)

10. (canceled)

11. The method as recited in claim 1, further comprising removing the two boron atoms from the dual boron-doped 2D graphitic carbon nitride catalyst following separation of the urea from the dual boron-doped 2D graphitic carbon nitride catalyst resulting in a two-dimensional graphitic carbon nitride scaffold that can be reused in the method of synthesizing urea.
